(19) United States
(12) Patent Application Publication
(10) Pub. No.: US 2018/0315510 A1
(43) Pub. Date: Nov. 1, 2018

(54) USE OF CLINICAL KNOWLEDGE TO IMPROVE USE OF NEXT GENERATION SEQUENCING

(71) Applicant: KONINKLIJKE PHILIPS N.V., Eindhoven (NL)

(72) Inventor: Brian David GROSS, North Andover, MA (US)

(21) Appl. No.: 15/963,144

(22) Filed: Apr. 26, 2018

Related U.S. Application Data
(60) Provisional application No. 62/490,767, filed on Apr. 27, 2017.

(51) Int. Cl. G16H 50/30; G06F 19/18; C12Q 1/6869

(52) U.S. Cl. CPC G16H 50/30; A61B 5/4848; C12Q 1/6869; G06F 19/18

(57) ABSTRACT

A method (100) of ensuring optimal use of next generation sequencing (NGS) in complex therapy decision making is disclosed herein. Such a method may include: identifying (105) an infected patient eligible for NGS; determining (110) a patient care trajectory for the infected patient, where this trajectory is determined from database records of physical contact by the infected patient with a healthcare resource; sequencing (115) an isolate from the infected patient; while sequencing, identifying (120) additional patients at risk of infection, determining (125) overlap in the patient care trajectory of the infected patient and patient care trajectories of additional patients, and determining (130) a risk of infection to the additional patients based on this overlap and clinical data points for the additional patients; determining (135) an updated risk of transmission to the additional patients; and causing (140) a computing device to render output of the updated risk of transmission to the one or more additional patients.

### CROSS-REFERENCE TO PRIOR APPLICATIONS

This application claims the benefit of U.S. Provisional Application No. 62/490,767, filed on 27 Apr. 2017. This application is hereby incorporated by reference herein.

### TECHNICAL FIELD

Various embodiments described herein are directed generally to health care. More particularly, but not exclusively, various methods and apparatus disclosed herein relate to ensuring optimal use of next generation sequencing in complex therapy decision making.

### BACKGROUND

The time required and accuracy of pathogen identification based on next generation sequencing (hereinafter “NGS”) impacts the clinical utility of utilizing NGS. Complex therapy decisions, such as whether to quarantine a patient or change a patient's antibiotic regimens, must be made quickly, and tradeoffs will likely need to be made and will also likely benefit an infected patient versus an overall goal of antibiotic stewardship.

Nosocomial infections, or hospital acquired infections, contribute to healthcare costs and poor clinical outcomes. By examining differences in quickly evolving regions of the genomes of an infectious organism, NGS technology has the capability to distinguish a pathogen not transmitted as part of an active healthcare encounter from a pathogen transmitted in a healthcare environment. Ideally, each suspected infection would be sequenced; however this is impractical, as a certain proportion of colonized hosts will be asymptomatic, and the cost of NGS is currently prohibitively high. The decision to sequence a pathogen or not impacts the cost and sensitivity of infection control surveillance activities. Accordingly, there is a need in the art to ensure that, given its high costs and latency, NGS is used effectively and efficiently for infectious disease control and monitoring.

### SUMMARY

The present application discloses one or more of the features recited in the appended claims and/or the following features which alone or in any combination, may comprise patentable subject matter. Techniques are described herein for ensuring optimal use of next generation sequencing in complex therapy decision making. In various embodiments, when a patient is determined to have an infection, a determination may be made, e.g., based on the patient's health/acuity and/or the patient's healthcare trajectory, of whether NGS is warranted. And if NGS is initiated, various techniques described herein may be performed to ensure that knowledge gained from the NGS is used as effectively and efficiently as possible.

In one aspect a method, implemented using one or more processors, includes: identifying an infected patient that is eligible for next-generation sequencing; determining, based on a hospital database, a patient care trajectory for the infected patient, where the patient care trajectory is determined from one or more database records of physical contact by the infected patient with a healthcare resource; sequencing an isolate from the infected patient; simultaneous to the sequencing, identifying one or more additional patients at risk of infection, where the identifying includes: determining, based on the hospital database, overlap in the patient care trajectory of the infected patient and one or more additional patient care trajectories of the one or more additional patients, and determining a risk of infection to the one or more additional patients based on the overlap and a plurality of clinical data points for each of the one or more additional patients; determining, based on sequence data from the isolate sequenced and the risk of infection to the one or more additional patients, an updated risk of transmission to the one or more additional patients; and causing one or more computing devices to render output that includes a user interpretable representation of the updated risk of transmission to the one or more additional patients.

In some embodiments, the healthcare resource includes one or more of a unit, a bed, or a procedure room. In other embodiments, the healthcare resource includes one or more caregivers in contact with the infected patient. In still other embodiments, the healthcare resource includes one or more pieces of healthcare equipment used by the infected patient or medical personnel to treat the infected patient.

In some embodiments, the plurality of clinical data points for each of the one or more additional patients includes one or more of a group consisting of: age, sex, immunological frailty, type of admission, current antibiotic use, lifetime antibiotic use, or medical history. In other embodiments, the plurality of clinical data points for each of the one or more additional patients includes one or more real-time physiological parameters. In some embodiments, the one or more real-time physiological parameters includes one or more of a group consisting of: blood pressure, heart rates, blood oxygenation, or temperature.

In some embodiments, determining the updated risk of transmission to the one or more additional patients includes evaluating a virulence level of the isolate. In other embodiments, determining the updated risk of transmission to the one or more additional patients includes evaluating an antibiotic resistance profile of the isolate.

In some embodiments, the method further comprising displaying a user interpretable representation of one or more proposed treatment protocol modifications for the one or more patients. In other embodiments, the user interpretable representation of the updated risk of transmission to the one or more additional patients is a heat map.

In another aspect a method of using clinical knowledge to optimize real-time next-generation sequencing is disclosed, where the method is implemented using one or more processors. The method includes: identifying an infected patient that is eligible for next-generation sequencing; determining, based on a hospital database, a patient care trajectory for the infected patient, where the patient care trajectory is determined from one or more database records of physical contact by the infected patient with a healthcare resource; sequencing an isolate from the infected patient; simultaneous to the sequencing, identifying one or more additional patients at risk of infection, the identifying including: determining, based on the hospital database, overlap in the patient care trajectory of the infected patient and one or more additional patient care trajectories of the one or more additional patients, and determining a risk of infection to the one or more additional patients based on the overlap and a plurality of clinical data points for each of the one or more additional patients; determining, based on sequence data from the isolate sequenced and the risk of infection to the one or more additional patients, an updated risk of transmission to the one or more additional patients, where the sequence data includes information about an virulence level of the isolate and an antibiotic resistance profile of the isolate; and causing one or more computing devices to render output that includes a user interpretable representation of the updated risk of transmission to the one or more additional patients and one or more proposed treatment protocol modifications for the one or more patients.

In another aspect at least one non-transitory computer-readable medium including instructions that, in response to execution of the instructions by one or more processors, cause the one or more processors to perform operations are disclosed. The operations including: determining, based on a hospital database, a patient care trajectory for an infected patient, where the patient care trajectory is determined from one or more database records of physical contact by the infected patient with a healthcare resource; identifying one or more additional patients at risk of infection, where the identifying includes: determining, based on the hospital database, overlap in the patient care trajectory of the infected patient and one or more additional patient care trajectories of the one or more additional patients, and determining a risk of infection to the one or more additional patients based on the overlap and a plurality of clinical data points for each of the one or more additional patients; determining, based on sequence data from an isolate sequenced and the risk of infection to the one or more additional patients, an updated risk of transmission to the one or more additional patients; and causing one or more computing devices to render output that includes a user interpretable representation of the updated risk of transmission to the one or more additional patients or one or more proposed treatment protocol modifications for the one or more patients.

In some embodiments, the healthcare resource includes one or more of a unit, a bed, a procedure room, one or more caregivers in contact with the infected patient, or one or more pieces of healthcare equipment used by the infected patient.

In some embodiments, the plurality of clinical data points for each of the one or more additional patients includes one or more of a group consisting of: age, sex, immunological frailty, type of admission, current antibiotic use, lifetime antibiotic use, or medical history. In other embodiments, the plurality of clinical data points for each of the one or more additional patients includes one or more real-time physiological parameters selected from a group consisting of: blood pressure, heart rates, blood oxygenation, or temperature.

In some embodiments, determining the risk of transmission to the one or more additional patients includes evaluating a virulence level of the isolate. In other embodiments, determining the risk of transmission to the one or more additional patients includes evaluating an antibiotic resistance profile of the isolate.

In some embodiments, the user interpretable representation of the updated risk of transmission to the one or more additional patients is a heat map.

### BRIEF DESCRIPTION OF THE DRAWINGS SUMMARY

In the drawings, like reference characters generally refer to the same parts throughout the different views. Also, the drawings are not necessarily to scale, emphasis instead generally being placed upon illustrating various principles of the embodiments described herein.

FIG. 1 depicts an exemplary method of using clinical knowledge to optimize real-time NGS, in accordance with various embodiments described herein.

FIG. 2 illustrates an exemplary hardware diagram 200 for implementing a sequencer, and/or a device for processing data received from a sequencer, in accordance with various embodiments described herein.

FIG. 3 illustrates an exemplary a user interpretable representation in the form of a visual representation, in accordance with various embodiments described herein.

FIG. 4 illustrates an exemplary user interpretable representation in the form of a heat map, in accordance with various embodiments described herein.

### DETAILED DESCRIPTION

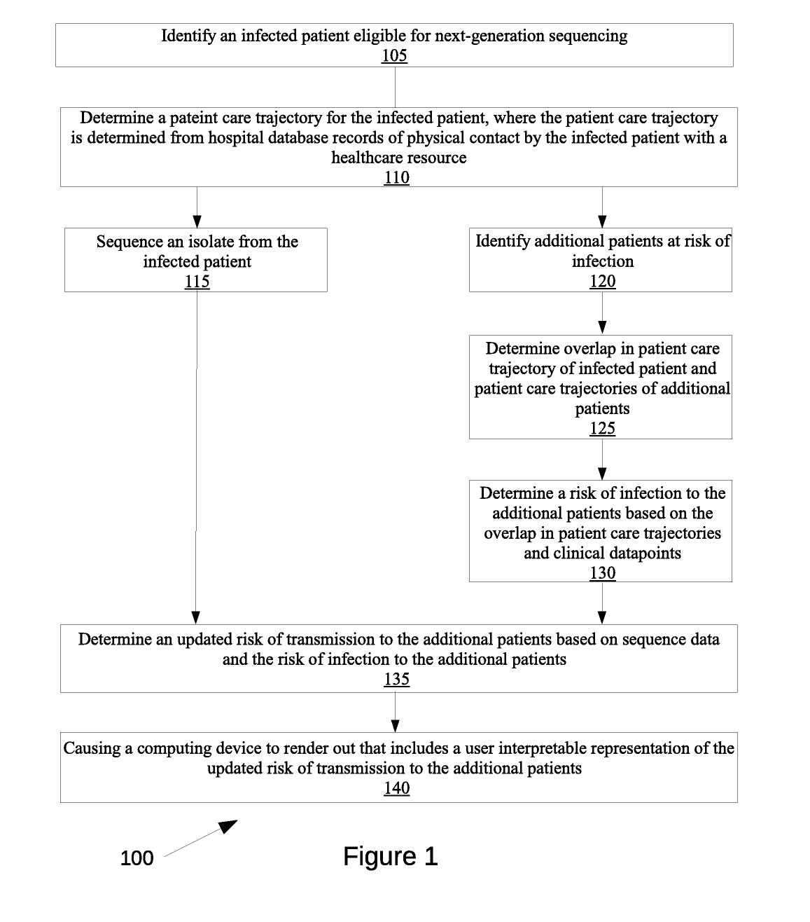

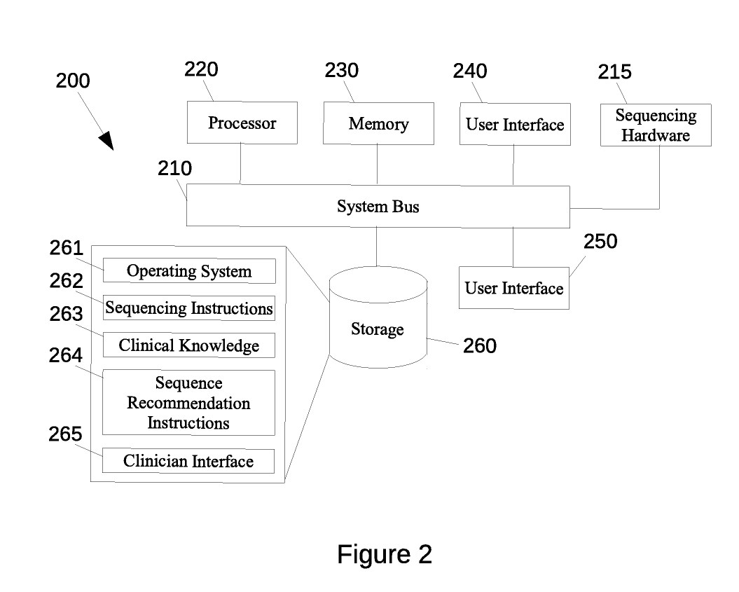

Various methods of using clinical knowledge to optimize real-time NGS are described herein. FIG. 1 illustrates a flowchart of an exemplary method 100 described herein. In some instances, these methods may begin with an identification 105 of an infected patient that is eligible for next-generation sequencing. Typically, when a clinical user (e.g. physician) orders culturing, a hospital system may review that particular patient's clinical data pursuant to computer interpretable guidelines (CIG) definitions for a particular infection's treatment and risk definitions in order to determine whether to recommend sequencing based on an infection risk (e.g. patient age, gender, symptomology, prior history, prior antibiotic use, and other input such as a positive culture or other tests). For example, if a clinician orders a urine culture and sensitivity the hospital system may review that particular patient's clinical data pursuant to CIG definitions for urinary tract infections (UTIs). Based on the results of the culture and/or other microbiological testing (e.g. bioMérieux's API®) and/or the results of the review of the patient's clinical data, the system determines whether or not an isolate from the infected patient should be sequenced. This determination, regarding whether to sequence or not, is described in greater detail with respect to FIG. 2 and the sequence recommendation instructions 264. Although, described with respect to a urine culture and sensitivity, this is not intended to be limiting, as the identification of an infection eligible for NGS is not limited to UTIs, and may be one or more of any other types of infection.

At block 110, a patient care trajectory for the infected patient may be determined based on a hospital database; for example, a database of clinical knowledge such as illustrated in FIG. 2 that includes a variety of clinical correlate information may be used. The patient care trajectory may be determined from one or more database records of physical contact by the infected patient with what will be referred to herein as a “healthcare resource.” Accordingly, in some embodiments, a “patient trajectory” may include a list of medical resources with which the patient had physical contact.” A patient trajectory may include various levels of granularity, such as times of contact with each healthcare resource, number of contacts with each healthcare resource, and so forth.

In various embodiments, a “healthcare resource” may be a location, such as a unit or ward of a clinical care facility, a bed or room number, a procedure room, or any other location with which the infected patient may have been in physical proximity. Additionally or alternatively, a healthcare resource may be one or more caregivers (e.g. physicians, nurses, certified nursing assistants, respiratory therapists, occupational therapists, physical therapists, phlebotomists, or the like). Additionally or alternatively, a healthcare resource may be one or more pieces of healthcare equipment used by the infected patient or by a caregiver to treat the infected patient. For example this equipment may include, but is not limited to: endoscopes; dialysis machines; ventilators; incubators; respiratory therapy equipment; thermometers; various patient monitoring equipment; blood pressure cuffs; ultrasound equipment; glucometers; and so on. It should be understood, that the preceding is not an exhaustive list of possible equipment, and that there may be many other types of equipment may be used by and/or to treat a patient.

At block 115, an isolate from the infected patient may then be sequenced using NGS technology. In some embodiments, where the hospital or clinical care environment does not have a sequencer, such an isolate (or genetic material therefrom) from the infected patient may be sent to a separate sequencing facility. In other embodiments, the hospital or clinical care facility may have their own sequencer and the isolate may be sequenced in-house.

While an isolate from the infected patient is being sequenced (block 115), at block 120 one or more additional patients at risk of contracting the infection may be identified. These one or more additional patients may not be known to be currently infected with the same organism as the infected patient, and therefore may also be referred to as “non-infected” patients. This identification includes, at block 125, examining one or more hospital databases for overlap in the patient trajectory of the infected patient with patient trajectories of one or more additional patients. This overlap may come in the form of the any number of potential commonalities. For example, the one or more additional patients may have been located on the same unit/ward of the clinical care facility at the same time; the one or more additional patients may have been cared for by the same caregiver; and/or the one or more additional patients may have used the same piece of medical equipment. The preceding are merely illustrative examples and are not intended to be limiting.

At block 130, a risk of infection is determined, based on the overlap in patient care trajectories, as well as one or more clinical data points for each the additional patients identified. In some embodiments, such clinical data points may include patient demographic information and medical history, such as patient age, sex, height, weight, type of admission, current antibiotic usage, lifetime antibiotic usage, and/or a measure of immunological frailty (e.g. white blood cell count, T-cell count, HIV status, or the like). In other embodiments, such clinical data points may include one or more real-time physiological parameters, such as blood pressure, heart rate, blood oxygenation, and/or temperature.

More specifically, in some embodiments, the risk of infection to the one or more additional patients may be determined through use of a trained model (e.g., regression model, neural network, support vector machine, etc.) that accepts various features stored in or derived from the patient care trajectories and/or any of the clinical data points previously discussed herein in order to determine a risk of infection to the one or more additional patients. In such embodiments, the trained model may be trained using information obtained from historic epidemics and historic outcomes from those epidemics, either within the same hospital or clinical environment or in other hospitals or clinical environments. In other embodiments, the risk of infection to the one or more additional patients may be determined through use of one or more predetermined algorithms. Regardless of how it is determined, the risk of infection to one or more patients is a risk assessment for each of the one or more patients of how likely each of those patients are to be infected by the infected patient (either directly or through indirect contact).

Once the sequencing is complete, sequence data may be analyzed and may provide additional information about the cause of the infection. For example, in some embodiments, the sequence data may include information regarding the antibiotic resistance (e.g. presence of plasmid mediated antibiotic resistance, antibiotic resistance mutations, and/or the like) of the organism. In other embodiments, the sequence data may include information regarding the virulence and/or transmissibility of the organism. At block 135, this sequence data, in combination with the risk of infection determined at block 130, may be analyzed together in order to determine an updated infection risk. This updated risk of transmission includes analysis of both organism-specific information (e.g. sequence data such as virulence, antibiotic resistance, and/or the like) as well as patient-specific information in order to determine which patients may be most at risk of acquiring the infection via transmission from the infected patient.

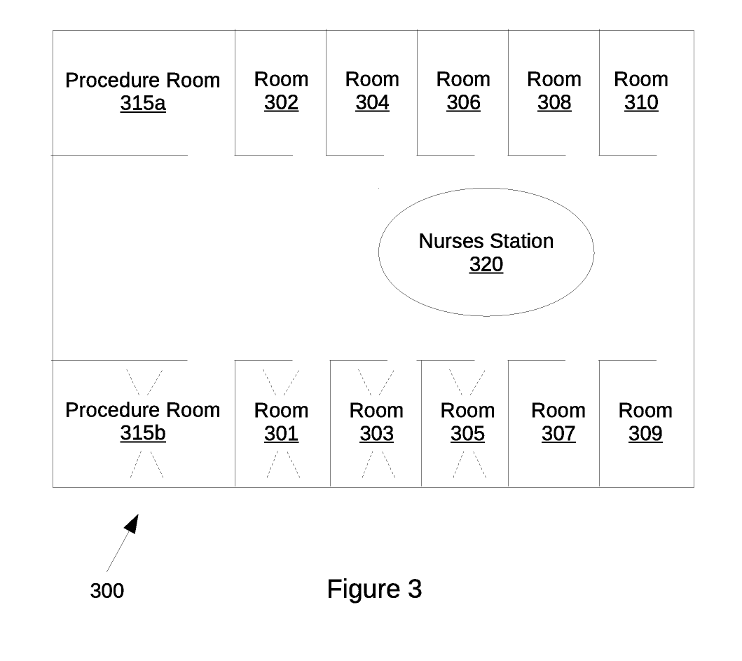

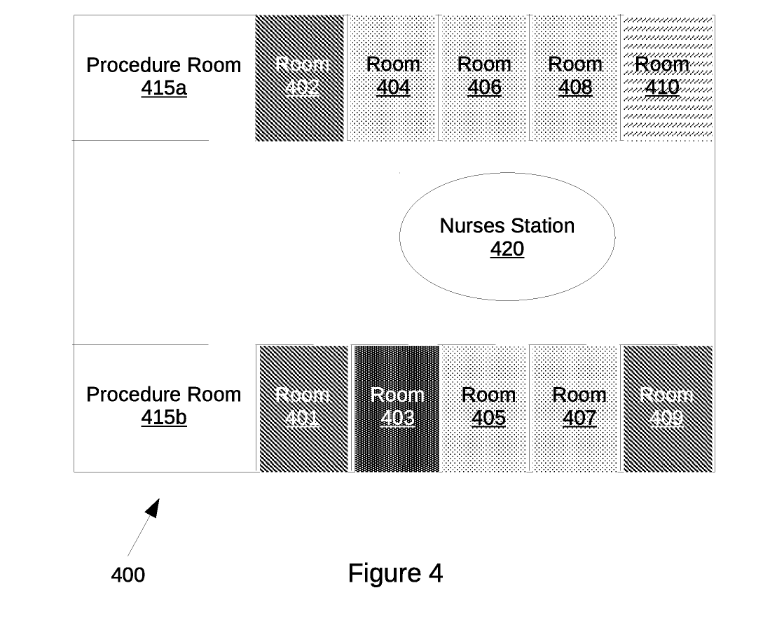

At block 140, a computing device may cause an output to be displayed to a user, where the display may include a user interpretable representation of the updated risk of transmission. In some embodiments, the computing device may be a desktop computer, laptop computer, server, mobile computing device (e.g. smartphone, tablet, or the like) and/or any other form of computing device known in the art. In some embodiments, the user interpretable representation may include a list of potential actions to prevent further spread of the infection and/or provide treatment options for the one or more additional patients who may have been exposed to the infection. For example, some possible actions that may be presented to the clinician may include: “isolate Mr. Infected Patient;” “change Ms. Smith's antibiotic to antibiotic X;” “increase frequency of vitals monitoring for Mr. Doe;” and so on. In other embodiments, this may be visually represented, by a map of a clinical care environment (such as illustrated in FIG. 3), where a notification of a recommendation for particular patients may be provided and indicated by a visual marker (e.g. a flashing light, different color indicator, etc.) that prompts the user to examine the user interpretable representations for each patient. In still other embodiments, the user interpretable representations presented to a user may be in the form of a heat map, which is discussed in greater detail with respect to FIG. 4.

The method of Figure is not meant to be limiting, and various operations may be added, omitted, and/or reordered. As one example, in some embodiments, the operations of block 115 (sequencing an isolate) may be performed conditionally, e.g., based on determinations from blocks 120-130 that there are, in fact, other patients with healthcare trajectories that overlapped with the infected patient's healthcare trajectory. As noted previously, NGS is costly, and therefore it may be beneficial to refrain from initiating NGS sequencing (block 115) if there is insufficient risk that other patients might also be infected. This might be the case, for instance, if the healthcare resources the infected patient interacted with were thoroughly sterilized before coming into contact with other patients.

FIG. 2 illustrates an exemplary hardware diagram 200 for implementing a sequencer, and/or a device for processing data received from a sequencer (particularly in instances where the clinical care facility does not have its own sequencer). As shown, the device 200 includes a processor 220, memory 230, user interface 240, communication interface 250, and storage 260 interconnected via one or more system buses 210. In some embodiments, such as those where the hardware implements a sequencer, the hardware may include additional sequencing hardware 215 such as, for example, a pore-based sequencer. It will be understood that FIG. 2 constitutes, in some respects, an abstraction and that the actual organization of the components of the device 200 may vary and may also be more complex than illustrated.

The processor 220 may be any hardware device capable of executing instructions stored in memory 230 or storage 260 or otherwise processing data. As such, the processor may include a microprocessor, field programmable gate array (FPGA), application-specific integrated circuit (ASIC), or other similar devices.

The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. It will be apparent that, in embodiments where the processor includes one or more ASICs (or other processing devices) that implement one or more of the functions described herein in hardware, the software described as corresponding to such functionality in other embodiments may be omitted.

The user interface 240 may include one or more devices for enabling communication with a user such as an administrator. For example, the user interface 240 may include a display, a mouse, and a keyboard for receiving user commands. In some embodiments, the user interface 240 may include a command line interface or graphical user interface that may be presented to a remote terminal via the communication interface 250.

The communication interface 250 may include one or more devices for enabling communication with other hardware devices. For example, the communication interface 250 may include a network interface card (NIC) configured to communicate according to the Ethernet protocol. Additionally, the communication interface 250 may implement a TCP/IP stack for communication according to the TCP/IP protocols. Various alternative or additional hardware or configurations for the communication interface 250 will be apparent.

The storage 260 may include one or more machine-readable storage media such as read-only memory (ROM), random-access memory (RAM), magnetic disk storage media, optical storage media, flash-memory devices, or similar storage media. In various embodiments, the storage 260 may store instructions for execution by the processor 220 or data upon with the processor 220 may operate. For example, the storage 260 may store a base operating system 261 for controlling various basic operations of the hardware 200. In instances where the hardware 200 implements a sequencer (and includes sequencing hardware 215), the storage 260 may also include sequencing instructions 262 for operating the sequencing hardware 215 and receiving commands from other software (e.g., commands to eject a strand to waste or staging, reverse a strand, configure the pore matrix, reread a region, etc.). Furthermore, the storage 260 may also store clinical knowledge 263 such as NGS pathogen information for the site (including current and historic clinical knowledge), clinical correlate information for both infected and non-infected patients (such as the information discussed in detail below), multi-encounter host information (e.g., lifetime antibiotic use, and clinical information including outcomes), real-time computerized physical order entry and electronic medical record information, and the like.

Sequence recommendation instructions 264 may be configured to analyze the clinical knowledge and generate a recommendation (e.g., to be presented via the user interface) as to whether to order pathogen or other sequencing for the patient (see generally block 105 of FIG. 1). In various embodiments, the sequence recommendation instructions 264 may include a trained model (e.g., regression model, neural network, Deep Learning network, etc.) that accepts various features stored in or derived from the clinical knowledge 264 and outputs a recommendation such as a binary indicator or score (e.g., on a scale of 10 or 100) indicating whether the system indicates that sequencing would be helpful and/or cost-effective. In some embodiments, the trained model may be trained using a machine learning algorithm (e.g., gradient descent) based on a dataset including features from previous patients and labels (e.g., as manually provided by the physician, automatically generated based on sequencing orders observed and eventual patient outcomes, or otherwise provided) of whether sequencing was appropriate or otherwise recommended to order.

It will be apparent that various information described as stored in the storage 260 may be additionally or alternatively stored in the memory 230. In this respect, the memory 230 may also be considered to constitute a “storage device” and the storage 260 may be considered a “memory.” Various other arrangements will be apparent. Further, the memory 230 and storage 260 may both be considered to be “non-transitory machine-readable media.” As used herein, the term “non-transitory” will be understood to exclude transitory signals but to include all forms of storage, including both volatile and non-volatile memories.

While the host device 200 is shown as including one of each described component, the various components may be duplicated in various embodiments. For example, the processor 220 may include multiple microprocessors that are configured to independently execute the methods described herein or are configured to perform steps or subroutines of the methods described herein such that the multiple processors cooperate to achieve the functionality described herein. Further, where the device 200 is implemented in a cloud computing system, the various hardware components may belong to separate physical systems. For example, the processor 220 may include a first processor in a first server and a second processor in a second server.

Referring now to FIG. 3, an embodiment of a visual representation of a user interpretable representation is illustrated. As illustrated, the visual representation may be in the form of a clinical care environment 300, such as a hospital unit or ward. Such a clinical care environment may include a plurality of patient rooms 301-310, a nurses' station 320, and/or one or more procedure rooms 315a, 315b. It is to be understood that a clinical care environment is not limited to those location illustrated in FIG. 3, and may include any number of additional spaces (e.g. operating rooms, waiting rooms, and so on). Furthermore, it is to be understood that they layout present in FIG. 3 is merely exemplary, and that clinical care environments may have any number of physical layouts. In some embodiments, a visual marker may indicate to a user (e.g. a clinician) that there may be recommendations for a particular patient and/or a particular location. For example, dashed lines in FIG. 3 represent a flashing light and/or flashing text to draw a user's attention to a particular location and prompt the user to click, touch, etc. the location which may bring up one or more potential actions for that particular patient(s) and/or location. The visual representation is not limited to a flashing light or text. The visual representation may be any number of other symbols, colors, etc. that indicate to a user that there is additional information for their review. In some embodiments, the visual representation may be incorporated into an existing display system for monitoring patients, such as those typically found at nurses' stations.

As a purely illustrative example, Rooms 301, 303, and 305, as well as Procedure Room 315b have a visual marker, the dashed line representing a flashing light and/or text, indicating a potential action for user review. The infected patient, Mr. Infected Patient, may have been located in Room 303, and a recommended action may be to isolate Mr. Infected Patient. The overlap in patient care trajectories of the Mr. Infected Patient and other patients may show that the same nurse that cared for Mr. Infected Patient in Room 303, also cared for Mr. Doe in Room 301 and Ms. Smith in Room 305, and as such there may be recommended actions for both Mr. Doe and Ms. Smith. Mr. Doe may be particularly immunologically frail and may already be on antibiotic X; however, the sequence data may indicate the isolate sequenced from Mr. Infected Patient is resistant to antibiotic X. Therefore, the recommended action may be to change Mr. Doe's antibiotic to Antibiotic Y; it may also be recommended to increase the frequency of monitoring of Mr. Doe's vitals. Ms. Smith may be in relatively good health, and therefore the recommended action for Ms. Smith, based on her potential exposure to the infection, may just be an increase in monitoring. Mr. Infected Patient may have also have had a procedure performed in Procedure Room 315b, and therefore the recommended action may be for an additional cleaning of all equipment within Procedure Room 315b.

Referring now to FIG. 4, another embodiment of a user interpretable representation is illustrated. Similar to the embodiment illustrated in FIG. 3, the representation may be in the form of a clinical care environment 400, such as a hospital unit or ward. Also similar to FIG. 3, the clinical care environment may include a plurality of patient rooms 401-410, a nurses' station 420, and/or one or more procedure rooms 415a, 415b. It is to be understood that a clinical care environment is not limited to those location illustrated in FIG. 4, and may include any number of additional spaces (e.g. operating rooms, waiting rooms, and so on). Furthermore, as with FIG. 3, it is to be understood that they layout present in FIG. 4 is merely exemplary, and that clinical care environments may have any number of physical layouts. The user interpretable representation of the embodiment illustrated in FIG. 4 is in the form of a heat map, where a user is presented with a visual indication of an updated risk of transmission (see block 140) for each patient. This updated risk of transmission factors in both organism-specific information (e.g. sequence data such as virulence, transmissibility, antibiotic resistance, and/or the like) as well as patient-specific information in order to determine which patients may be most at risk of acquiring the infection via transmission from the infected patient.

In FIG. 4, the shade of each patient's room provides a visual representation of the updated risk of transmission to that patient, for example the darker the shade, the more likely transmission. In FIG. 4, the patient in Room 403 may be the infected patient, indicated by the darkest intensity of the shading. The patients in Rooms 401, 402, and 409 have the highest likelihood of acquiring the infection based on the updated transmission risk. For example, this may mean that the patients in these rooms may have shared one or more caregivers and/or pieces of equipment with the infected patient, or that based on their individual health histories, immunological fragility, etc. these patients may be more prone to infection. Furthermore, the patient in Room 410 also has an increased risk of acquiring the infection, which illustrates that the increased risk of infection does not necessarily correlate with physical proximity to the infected patient. Although illustrated in FIG. 4 as shades of grey, this is not intended to be limiting, as a heat map may also utilize colors to indicate likelihood of transmission. For example, in some embodiments, shades of red may indicate a high risk of infection transmission to that patient, shades of yellow may indicate moderate risk of infection transmission to that patient, while shades of green may indicate a low risk of transmission to that patient.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

What is claimed is:

1. A method implemented using one or more processors and comprising:
identifying an infected patient that is eligible for next-generation sequencing;
determining, based on a hospital database, a patient care trajectory for the infected patient, wherein the patient care trajectory is determined from one or more database records of physical contact by the infected patient with a healthcare resource;
sequencing an isolate from the infected patient;
simultaneous to the sequencing, identifying one or more additional patients at risk of infection, wherein the identifying includes:
determining, based on the hospital database, overlap in the patient care trajectory of the infected patient and one or more additional patient care trajectories of the one or more additional patients, and
determining a risk of infection to the one or more additional patients based on the overlap and a plurality of clinical data points for each of the one or more additional patients;
determining, based on sequence data from the isolate sequenced and the risk of infection to the one or more additional patients, an updated risk of transmission to the one or more additional patients; and
causing one or more computing devices to render output that includes a user interpretable representation of the updated risk of transmission to the one or more additional patients.

2. The method of claim 1, wherein the healthcare resource includes one or more of a unit, a bed, or a procedure room.

3. The method of claim 1, wherein the healthcare resource includes one or more caregivers in contact with the infected patient.

4. The method of claim 1, wherein the healthcare resource includes one or more pieces of healthcare equipment used by the infected patient or medical personnel to treat the infected patient.

5. The method of claim 1, wherein the plurality of clinical data points for each of the one or more additional patients includes one or more of a group consisting of: age, sex, immunological frailty, type of admission, current antibiotic use, lifetime antibiotic use, or medical history.

6. The method of claim 1, wherein the plurality of clinical data points for each of the one or more additional patients includes one or more real-time physiological parameters.

7. The method of claim 6, wherein the one or more real-time physiological parameters includes one or more of a group consisting of: blood pressure, heart rates, blood oxygenation, or temperature.

8. The method of claim 1, wherein determining the updated risk of transmission to the one or more additional patients includes evaluating a virulence level of the isolate.

9. The method of claim 1, wherein determining the updated risk of transmission to the one or more additional patients includes evaluating an antibiotic resistance profile of the isolate.

10. The method of claim 1 further comprising displaying a user interpretable representation of one or more proposed treatment protocol modifications for the one or more patients.

11. The method of claim 1, wherein the user interpretable representation of the updated risk of transmission to the one or more additional patients is a heat map (400).

12. At least one non-transitory computer-readable medium comprising instructions that, in response to execution of the instructions by one or more processors, cause the one or more processors to perform the following operations:
determining, based on a hospital database, a patient care trajectory for an infected patient, wherein the patient care trajectory is determined from one or more database records of physical contact by the infected patient with a healthcare resource;
identifying one or more additional patients at risk of infection, wherein the identifying includes:
determining, based on the hospital database, overlap in the patient care trajectory of the infected patient and one or more additional patient care trajectories of the one or more additional patients, and
determining a risk of infection to the one or more additional patients based on the overlap and a plurality of clinical data points for each of the one or more additional patients;
determining, based on sequence data from an isolate sequenced and the risk of infection to the one or more additional patients, an updated risk of transmission to the one or more additional patients; and
causing one or more computing devices to render output that includes a user interpretable representation of the updated risk of transmission to the one or more additional patients or one or more proposed treatment protocol modifications for the one or more patients.

13. The at least one non-transitory computer-readable medium of claim 12, wherein the healthcare resource includes one or more of a unit, a bed, a procedure room, one or more caregivers in contact with the infected patient, or one or more pieces of healthcare equipment used by the infected patient.

14. The at least one non-transitory computer-readable medium of claim 12, wherein the plurality of clinical data points for each of the one or more additional patients includes one or more of a group consisting of: age, sex, immunological frailty, type of admission, current antibiotic use, lifetime antibiotic use, or medical history.

15. The at least one non-transitory computer-readable medium of claim 12, wherein the plurality of clinical data points for each of the one or more additional patients includes one or more real-time physiological parameters selected from a group consisting of: blood pressure, heart rates, blood oxygenation, or temperature.

16. The at least one non-transitory computer-readable medium of claim 12, wherein determining the risk of transmission to the one or more additional patients includes evaluating a virulence level of the isolate.

17. The at least one non-transitory computer-readable medium of claim 12, wherein determining the risk of transmission to the one or more additional patients includes evaluating an antibiotic resistance profile of the isolate.

18. The at least one non-transitory computer-readable medium of claim 12, wherein the user interpretable representation of the updated risk of transmission to the one or more additional patients is a heat map.

19. A system, comprising:
one or more processors; and
memory configured to store instructions that, when executed by the one or more processors, cause the one or more processors to perform operations that include:
identifying an infected patient that is eligible for next-generation sequencing;
determining, based on a hospital database, a patient care trajectory for the infected patient, wherein the patient care trajectory is determined from one or more database records of physical contact by the infected patient with a healthcare resource;
sequencing an isolate from the infected patient;
simultaneous to the sequencing, identifying one or more additional patients at risk of infection, wherein the identifying includes:
determining, based on the hospital database, overlap in the patient care trajectory of the infected patient and one or more additional patient care trajectories of the one or more additional patients, and
determining a risk of infection to the one or more additional patients based on the overlap and a plurality of clinical data points for each of the one or more additional patients;
determining, based on sequence data from the isolate sequenced and the risk of infection to the one or more additional patients, an updated risk of transmission to the one or more additional patients, wherein the sequence data includes information about an virulence level of the isolate and an antibiotic resistance profile of the isolate; and
causing one or more computing devices to render output that includes a user interpretable representation of the updated risk of transmission to the one or more additional patients and one or more proposed treatment protocol modifications for the one or more patients.

20. The system of claim 19, wherein the plurality of clinical data points for each of the one or more additional patients includes one or more of a group consisting of: age, sex, immunological frailty, type of admission, current antibiotic use, lifetime antibiotic use, or medical history.
